SEQUENCE LISTING (10 sequences)

SEQ ID NO: 1 - DNA, 1083 bp, Homo sapiens
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctga

SEQ ID NO: 2 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 3 - protein, 606 aa, Artificial Sequence
  Synthetic Peptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGNGGNASMSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 4 - protein, 605 aa, Artificial Sequence
  Synthetic Peptide
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMESLDGNGGNGSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - protein, 606 aa, Artificial Sequence
  Synthetic Peptide
MVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYKGNGGNASMSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 6 - protein, 605 aa, Artificial Sequence
  Synthetic Peptide
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMESLDGNGGNGSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 7 - DNA, 1818 bp, Artificial Sequence
  Synthetic Oligonucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaatggcggcaatgctagcatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcc

SEQ ID NO: 8 - DNA, 1818 bp, Artificial Sequence
  Synthetic Oligonucleotide
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtccctcgacggcaatggcggcaatggcagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 1818 bp, Artificial Sequence
  Synthetic Oligonucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaatggcggcaatgctagcatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcc

SEQ ID NO: 10 - DNA, 1818 bp, Artificial Sequence
  Synthetic Oligonucleotide
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtccctcgacggcaatggcggcaatggcagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2008/0227131 A1
(43) Pub. Date: Sep. 18, 2008

(54) Signalling Assay and Cell Line

(75) Inventors: Simon L. J. Stubbs, Little Chalfont (GB); Catherine Hather, Little Chalfont (GB); Sandra Ross, Ventura, CA (US); David Powers, Newbury Park, CA (US)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE UK LIMITED, LITTLE CHALFONT (GB)

(21) Appl. No.: 12/091,579

(22) PCT Filed: Oct. 26, 2006
(86) PCT No.: PCT/US06/60251

§ 371 Date: Apr. 25, 2008

Related U.S. Application Data
(60) Provisional application No. 60/731,364, filed on Oct. 28, 2005.

(51) Int. Cl. C12Q 1/48; C07K 14/00; C07H 21/00; C12N 15/63; C12N 5/10

(52) U.S. Cl. 435/15; 530/402; 536/23.4; 435/320.1; 435/419; 435/348; 435/349; 435/325; 435/366

(57) ABSTRACT

The invention relates to a sensor for indicating the transduction and inhibition of stress signals through the p38-MAPK pathway in living cells. The sensor comprises a reporter gene product and an isoform of p38 Mitogen Activated Protein Kinase (MAPK). The invention also provides plasmid and viral vectors containing nucleic acids encoding the sensor for the transfection of living cells. Stable cell lines expressing the sensor can be used in a live-cell or fixed-cell assay to measure activation or modulation of the pathway.

### CROSS REFERENCE TO RELATED APPLICATIONS

This application claims priority to U.S. provisional patent application No. 60/731,364 filed Oct. 28, 2005, the entire disclosure of which is incorporated herein by reference in its entirety.

### FIELD OF THE INVENTION

The present invention relates to a sensor for indicating the transduction and inhibition of stress signals through the p38 Mitogen Activated Protein Kinase pathway in living cells. Stable cell lines expressing the sensor are provided which can be used in a live-cell or fixed-cell assay to measure activation or modulation of the pathway.

### BACKGROUND OF THE INVENTION

Mitogen-activated protein kinases (MAPKs) are serine/threonine kinases that connect cell-surface receptors to regulatory targets within cells and convert extracellular signals into various cellular outputs. The p38 MAPK cascade is activated by stress or cytokines and transmits through a complex pathway of sequentially activating protein kinases leading to gene expression. The main activator kinases for the p38 MAPK pathway are MKK3 and MKK6, and they signal through the hub of the cascade (see for example the NCBI web site; Shi and Gaestel 2002, Biol Chem 282, 1519-36; Herlaar, E. and Brown, Z. (1999), Mol Med Today 5, 439-447; Ichijo, H. (1999) Oncogene 18, 6087-6093; Tibbles, L. A. and Woodgett, J. R. (1999) Cell Mol Life Sci. 55, 1230-1254).

The stress-activated protein kinase p38 isoforms comprise p38 alpha (p38α, or MAPK14; Han et al., 1993, J Biol Chem, 1993, 268, 25009-25014), p38 beta (p38ε; Jiang et al., 1996, J Biol Chem., 271, 17920-17926), stress-activated protein kinase 3/p38 gamma (p38γ or ERK6, SAPK3; Li et al., 1996, Biochem Biophys Res Comm, 228, 334-340) and stress-activated protein kinase 4/p38 delta (p38δ, SAPK4; Jiang et al., 1997, J Biol Chem, 272, 30122-30128). Each p38 isoform may have different biological functions and different biological substrates but they all phosphorylate substrates containing the minimal consensus sequence Ser/Thr-Pro (Kuma et al., 2005, J Biol Chem, 280, 19472-19479).

Unlike the three additional isoforms of p38 MAPK, the alpha isoform (MAPK14) has been exhaustively investigated, is present in all mammals and demonstrates ubiquitous expression throughout the tissues of the body (Zarubin , T and Han, J., 2005, Cell Res, 15, 11-18). Importantly, p38 MAPK acts as the primary hub for stress signalling; upstream stress related signals are funnelled down through p38 MAPK permitting control over downstream signal diversification. p38 MAPKs phosphorylate a wide range of regulatory proteins in vivo including the MAPKAPK family, STAT1, p53, SAP1, the C/EBP family, USF-1, NFAT, PPAG coactivator, CDC25B and others (see, for example, the Biocarta web site). Phosphorylation of such a diverse range of signalling proteins provides p38 with influence over the cell cycle, growth, differentiation, apoptosis, migration and cytoskeletal remodelling.

The desire to find and characterise novel therapeutic compounds and the importance of p38 MAPK in such diverse cellular responses has fuelled the search within the Pharmaceutical and Biotechnological industries for inhibitors and activators of the pathway. In cellular assays, the activation of p38α (MAPK14) can be detected directly via phospho-specific immunofluorescence assays (e.g. Rabbit anti-phospho-p38 MAPK polyclonal antibodies available from Zymed Laboratories San Francisco, USA; or p38 Activation Kit available from Cellomics, Inc., Pittsburgh, USA) or indirectly through measurement of the effects of the enzyme upon target molecules downstream in the signalling process such as MAPKAPK2 (GE Healthcare Bio-sciences, Amersham, UK).

Since p38 is regarded as a major control protein that funnels upstream signals and controls signal diversification downstream, a direct assay will provide precise data regarding the transduction of stress signals. An indirect assay will be less precise since a stress signal that is transduced through p38 MAPK may not be transduced, or the signal may be diluted, through a particular downstream protein. In addition, indirect assays can produce false-positive results due to off-target effects caused by the complexity, diversity and cross-communication of signal transduction pathways.

Direct immunofluorescence assays, however, are not homogeneous and cannot be conducted on living cells in real time. Moreover, the fixation process requires numerous washing and antibody treatment steps which can introduce artefacts and errors in the assay, making the resulting imaging data difficult to interpret. The screening of large numbers of compounds also requires a considerable amount of specific-antibody which is both resource and cost demanding. Furthermore, a phospho-specific immunodetection system provides a means to detect activation only via a specific phosphorylation event (e.g. phosphorylation of Thr180/Tyr182) and although a considerable amount of information is available on p38, alternative activation sites may yet be discovered.

US 2005/0118663 describes methods for identifying novel serine hydroxymethyltransferase (SHMT) modulators which have potential as anticancer compounds to control cell proliferation. Some of the methods disclosed in the document involve identifying agents that stimulate p38 kinase activity, as such compounds may inhibit SHMT enzymatic activity. Although the document alludes to the use of a p38 reporter gene cellular assay to screen test agents, there is no evidence that such assays have been developed as all test results are based upon in vivo labelling and antibody experiments.

US 2004/0124186 describes methods for screening for constitutively activated mutants of a desired eukaryotic MAPK pathway member of a MAPK pathway and for their use in screening for inhibitors of a MAPK pathway in drug design. The use of such activated mutants in a reporter gene assay is alluded to but there are no data to support that such assays were produced.

The production of a homogeneous, stable, p38 MAPK reporter gene assay in living cells has proved difficult because overexpression of p38 MAPK in mammalian cells has been shown to be cytotoxic and to induce apoptosis and senescence (Chen et al., 2003, Cell Death and Differentiation, 10, 516-527; Cong, F. and Goff, S., 1999, Proc Natl Acad Sci USA, 96, 13819-13824; Zarubin, T and Han, J., 2005, Cell Res, 15, 11-18). Consequently, there are no reports of such assays substantiated by convincing experimental data in the public domain.

Accordingly, it is an object of the present invention to provide a simple, homogeneous, cost-effective assay for p38α (MAPK14) which does not suffer from the problems associated with the prior art assays described above. Such assays are of particular interest to the Pharmaceutical and Biotechnological industries in their programmes to screen for therapeutic compounds, such as anticancer and pro- and anti-inflammatory compounds.

### Definitions

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds.

It will be appreciated that “proteins” often contain amino acids other than the amino acids commonly referred to as the naturally occurring amino acids, and that many amino acids, including the terminal amino acids, may be modified in a given protein, either by natural processes, such as processing and other post-translational modifications, or by chemical modification techniques which are well known to the art. Several particularly common modifications including glycosylation, lipid attachment, sulfation, gamma-carboxylation of glutamic acid residues, hydroxylation and ADP-ribosylation are described in most basic texts such as ‘Proteins—Structure and Molecular Properties, 2nd Ed., T. E. Creighton, W. H. Freeman and Company, New York, 1993; and Rattan et al., “Protein Synthesis: Posttranslational Modifications and Aging”, Ann. N.Y. Acad. Sci., 1992, 663: 48-62.

The modifications that occur in a protein often will be a function of how it is made. For proteins made by expressing a cloned gene in a host, for instance, the nature and extent of the modifications in large part will be determined by the host cell's posttranslational modification capacity and the modification signals present in the protein amino acid sequence. For instance, as is well known, glycosylation often does not occur in bacterial hosts such as E. coli. Accordingly, when glycosylation is desired, a polypeptide should be expressed in a glycosylating host, generally a eukaryotic cell. It will be appreciated that the same type of modification may be present in the same or varying degrees at several sites in a given polypeptide. Also, a given protein may contain many types of modifications. The term “protein” encompasses all such modifications, particularly those which result from expressing a polynucleotide in a host cell.

As used herein, the term “fusion protein” (or “chimeric protein”), is a non-naturally occurring protein which consists of two or more different protein sequences. For example, WO 03/087394 describes fusion proteins comprising a substrate and a serine/threonine kinase. The source of the different sequences can be from the same or different species or genus, or from synthetic, non-naturally occurring sequences. A fusion protein can have separate functions attributable to the different sequences, or different sequences can contribute to a single function. The fusion protein of the invention can be prepared in any suitable manner; such means are well known to those skilled in the art and are described in detail below.

The term “reporter gene product”, as used herein, refers to the detectable polypeptide which is encoded by a reporter gene. Such reporter genes are well known in the art and have been used to ‘report’ many different properties and events such as, for example, the strength of promoters , the efficiency of gene delivery systems, the intracellular fate of a gene product or the success of molecular cloning efforts. Examples of reporter genes include nitro reductase (NTR), chloramphenical acetyltransferase (CAT), β-galactosidase (GAL), β-glucoronidase (GUS), luciferase (LUC) and fluorescent proteins (FP).

“Isoform”, as used herein, refers to any of multiple forms of the same protein that differ in their primary structure but retain the same function.

The term “operably linked” refers to a functional relationship between two or more polynucleotides (e.g. DNA sequences). Typically it refers to the functional relationship of a transcriptional regulatory sequence to a transcribed sequence. For example, a promoter or enhancer sequence is operably linked to a coding sequence if it stimulates or modulates the transcription of the coding sequence in a suitable host cell.

A “nucleotide sequence” is a nucleic acid which is a polymer of nucleotides (e.g. A,C,T,U,G, etc. or naturally occurring or artificial nucleotide analogues). Either the given nucleic acid or the complementary nucleic acid can be determined from any specified polynucleotide sequence.

The term “nucleic acid,” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides.

A “vector” is a composition for facilitating introduction, replication and/or expression of a selected nucleic acid in a cell. Vectors include, e.g., plasmids, cosmids, viruses, YACs, bacteria, poly-lysine, etc. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

“Plasmids” generally are designated herein by a lower case p preceded and/or followed by capital letters and/or numbers, in accordance with standard naming conventions that are familiar to those of skill in the art. The plasmids disclosed herein are either commercially available, publicly available on an unrestricted basis, or can be constructed from available plasmids by routine application of well known published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill in the art from the present disclosure.

A “host cell,” as used herein, refers to a prokaryotic or eukaryotic cell that contains heterologous DNA that has been introduced into the cell by any means, e.g., electroporation, calcium phosphate precipitation, microinjection, transformation, plasmid transfection, viral infection, etc.

A “host cell stably transformed with a nucleotide sequence” (used interchangeably with “stable cell line”) refers to a host cell in which the nucleotide sequence has been stably integrated into the genomic DNA of the cell; the characteristic of the cells to express the protein encoded by the nucleotide sequence is transferred on cell division.

The term “modulate” refers to a change in the cellular level or other biological activities of a reference molecule. Modulation can be up-regulation (i.e., activation or stimulation) or down-regulation (i.e. inhibition or suppression). With respect to modulation of expression level, the change can arise from, for example, an increase or decrease in expression of a reference gene (e.g. reporter gene), stability of mRNA that encodes the reference protein, translation efficiency, or a change in post-translational modifications or stability of the protein. The mode of action can be direct, e.g., through binding to the reference protein or to genes encoding the reference protein. The change can also be indirect, e.g., through binding to and/or modifying (e.g., enzymatically) another molecule which otherwise modulates the reference protein.

The term “treating” has its normal meaning and refers to combining or contacting a first entity with a second entity (e.g. a cell with an agent).

The term “agent” includes any physical or chemical entity. An example of a physical entity is electromagnetic radiation (e.g. UV, IR). Where, for example, the agent is a chemical entity, it may be a substance, molecule, element, compound, or a combination thereof. It includes, but is not limited to, e.g., protein, polypeptide, small organic molecule, polysaccharide, polynucleotide, etc. It can be a natural product, a synthetic compound, or a chemical compound, or a combination of two or more substances. It can be a drug candidate with potential for therapeutic usage. Unless otherwise specified, the terms “agent”, “substance”, and “compound” can be used interchangeably.

### SUMMARY OF THE INVENTION

According to a first aspect of the invention, there is provided a fusion protein comprising a reporter gene product and an isoform of p38 Mitogen Activated Protein Kinase (MAPK).

Preferably, the fusion protein additionally comprises a linker group linking the reporter gene product to the p38 MAPK. Preferably, the linker group consists of a peptide comprising less than twenty, preferably less than fifteen, preferably less than ten peptides. More preferably, the linker group is a hepta peptide consisting of the amino acids GNGGNAS.

Suitably, the isoform of p38 MAPK is selected from the group consisting of p38-alpha (p38α, MAPK14), p38-beta (p38β), p38δ (SAPK4) and p38 gamma (p38γ or ERK6, SAPK3). Preferably, the isoform of p38 MAPK is p38-alpha (MAPK14).

Suitably, the reporter gene product is localisable by a detectable luminescent, fluorescent or radio-active moiety.

Suitably, the reporter gene product is a fluorescent protein such as a Green Flourescent Protein (GFP) derived from Aequoria Victoria, Renilla reniformis or other members of the class Anthozoa (Labas et al., Proc. Natl. Acad. Sci, (2002), 99, 4256-4261).

U.S. Pat. No. 6,172,188 describes variant GFPs wherein the amino acid in position 1 preceding the chromophorc has been mutated to provide an increase in fluorescence intensity. These mutants result in a substantial increase in the intensity of fluorescence of GFP without shifting the excitation and emission maxima. F64L-GFP has been shown to yield an approximate 6-fold increase in fluorescence at 37° C. due to shorter chromophore maturation time.

Preferably, the fluorescent protein is selected from the group consisting of Green Fluorescent Protein (GFP), Yellow Fluorescent Protein (YFP), Blue Fluorescent Protein (BFP), Cyan Fluorescent Protein (CFP), Red Fluorescent Protein (RFP), Enhanced Green Fluorescent Protein (EGFP) and Emerald.

Most preferably, the fluorescent protein is Enhanced Green Fluorescent Protein (Cormack, B. P. et al., Gene, (1996), 173, 33-38) or Emerald. EGFP has been optimised for expression in mammalian systems, having been constructed with preferred mammalian codons.

In a preferred embodiment, the fusion protein comprises Enhanced Green Fluorescent Protein and p38-alpha (MAPK14), e.g. SEQ ID NO: 3 (c-terminal EGFP-p38 alpha) or SEQ ID NO: 4 (n-terminal p38 alpha-EGFP).

In another preferred embodiment, the fusion protein comprises Emerald and p38 alpha (MAPK14), e.g. SEQ ID NO: 5 (c-terminal Emerald-p38 alpha) or SEQ ID NO: 6 (n-terminal p38 alpha-Emerald).

Preferably, the fusion protein is selected from the group consisting of SEQ ID NO: 3, SEQ ID NO: 4, SEQ ID NO: 5 and SEQ ID NO: 6.

It will be understood that the reporter gene product can be an enzyme. Suitable enzymes include nitro reductase (NTR), chloramphenical acetyltransferase (CAT), β-galactosidase (GAL), β-glucoronidase (GUS), alkaline phosphatase and luciferase (LUC).

In a second aspect of the present invention, there is provided a nucleotide sequence encoding a fusion protein as hereinbefore described. Thus, for example, SEQ ID NO: 3 is encoded by SEQ ID NO: 7, SEQ ID NO: 4 is encoded by SEQ ID NO: 8, SEQ ID NO: 5 is encoded by SEQ ID NO: 9, and SEQ ID NO: 6 is encoded by SEQ ID NO: 10.

Preferably, the nucleotide sequence is selected from the group consisting of SEQ ID NO: 7, SEQ ID NO: 8, SEQ ID NO: 9 and SEQ ID NO: 10.

Suitably, the nucleotide sequence is operably linked to a promoter, and is under the control of the promoter.

Preferably, the promoter is selected from the group consisting of mammalian constitutive promoter, mammalian regulatory promoter, human ubiquitin C promoter, viral promoter, SV40 promoter, CMV promoter, yeast promoter, filamentous fungal promoter and bacterial promoter. Preferably, where the promoter is a viral promoter, the promoter is either the CMV or the SV40 promoter. Most preferably, the promoter is the human ubiquitin C promoter.

According to a third aspect of the present invention, there is provided a replicable vector comprising a nucleotide sequence as hereinbefore described. Preferably, the vector is a plasmid vector.

When the vector is a viral vector, the vector is selected from the group consisting of cytomegalovirus, Herpes simplex virus, Epstein-Barr virus, Simian virus 40, Bovine papillomavirus, Adeno-associated virus, Adenovirus, Vaccina virus and Baculovirus vector.

In a fourth aspect of the present invention, there is provided a host cell transformed with a nucleotide sequence as hereinbefore described. Preferably, the host cell is stably transformed with a nucleotide sequence as hereinbefore described.

Suitably, the host cell is selected from the group consisting of plant, insect, nematode, bird, fish and mammalian cell. Preferably, the cell is a mammalian cell, most preferably a human cell. In one embodiment, the host cell of the fourth aspect is a human chondrosarcoma cell line SW1353.

Suitably, the host cell is capable of expressing the fusion protein as hereinbefore described.

According to a fifth aspect of the present invention, there is provided a method for detecting activation of p38 Mitogen Activated Protein Kinase (MAPK) in a living cell comprising the steps of
i) culturing a cell transformed to over-express a fusion protein as hereinbefore described;
ii) determining the localisation of the fusion protein within the cell with time;
   wherein a change in localisation of the fusion protein within the cell is indicative of activation.

In a sixth aspect of the present invention, there is provided a method for measuring the effect that an agent has upon activating p38 Mitogen Activated Protein Kinase (MAPK) in a living cell comprising the steps of
i) culturing a cell transformed to over-express a fusion peptide as hereinbefore described;
ii) determining the localisation of the peptide within the cell;
iii) treating the cell with the agent and determining the localisation of the peptide within the cell;
   wherein any difference in the localisation of the peptide within the cell relative to control cells untreated with the agent is indicative of the effect that the agent has upon activating p38 Mitogen Activated Protein Kinase (MAPK).

According to a seventh aspect of the present invention, there is provided a method for measuring the effect an agent has upon activating p38 Mitogen Activated Protein Kinase (MAPK) in a living cell comprising the steps of
i) culturing a first cell and a second cell which both over-express a fusion protein as hereinbefore described;
ii) treating the first cell with the agent and determining the localisation of the protein within the first cell;
iii) determining the localisation of the protein within the second cell which has not been treated with the agent;
   wherein any difference in the localisation of the protein within the first cell and second cell is indicative of the effect that the agent has upon activating p38 Mitogen Activated Protein Kinase (MAPK).

In an eighth aspect of the present invention, there is provided a method for measuring the effect an agent has upon activating p38 Mitogen Activated Protein Kinase (MAPK) in a living cell comprising the steps of
i) culturing a cell transformed to over-express a fusion protein as hereinbefore described;
ii) treating the cell with the agent and determining the localisation of the protein within the cell;
iii) comparing the localisation of the protein in the presence of the agent with a known value for the localisation of the protein in the absence of the agent;
   wherein any difference in the localisation of the protein within the cell in the presence of the agent and the known value in the absence of the agent is indicative of the effect that the agent has upon activating p38 Mitogen Activated Protein Kinase (MAPK).

According to a ninth aspect of the present invention, there is provided a method for measuring the effect that an agent has upon modulating the activation of p38 Mitogen Activated Protein Kinase (MAPK) in a living cell comprising the steps of
i) culturing a cell transformed to over-express a fusion peptide as hereinbefore described;
ii) treating the cell with the agent;
iii) treating the cell with a known activator of p38 Mitogen Activated Protein Kinase (MAPK) and determining the localisation of the peptide within the cell;
   wherein any difference in the localisation of the peptide within the cell relative to control cells untreated with the agent but treated with the known activator is indicative of the effect that the agent has upon modulating activation of p38 Mitogen Activated Protein Kinase (MAPK).

In a tenth aspect of the present invention, there is provided a method for measuring the effect an agent has upon modulating the activation of p38 Mitogen Activated Protein Kinase (MAPK) in a living cell comprising the steps of
i) culturing a first cell and a second cell which both over-express a fusion protein as hereinbefore described;
ii) treating the first cell with the agent;
iii) treating the first cell and the second cell with a known activator of p38 Mitogen Activated Protein Kinase (MAPK) and determining localisation of the protein within the first cell and the second cell;
   wherein any difference in the localisation of the protein within the first cell and second cell is indicative of the effect that the agent has upon modulating activation of p38 Mitogen Activated Protein Kinase (MAPK).

According to an eleventh aspect of the present invention, there is provided a method for measuring the effect an agent has upon modulating the activation of p38 Mitogen Activated Protein Kinase (MAPK) in a living cell comprising the steps of
i) culturing a cell transformed to over-express a fusion protein as hereinbefore described;
ii) treating the cell with the agent;
iii) treating the cell with a known activator of p38 Mitogen Activated Protein Kinase (MAPK) and determining the localisation of the peptide within the cell;
iv) comparing the localisation of the protein in the presence of the agent and the activator with a known value for the localisation of the protein in the presence of the activator but in the absence of the agent;
   wherein any difference in the localisation of the protein within the cell in the presence of the agent and said known value in the absence of the agent is indicative of the effect that the agent has upon modulating the activation of p38 Mitogen Activated Protein Kinase (MAPK).

Suitably, the known value of the method of the eighth and eleventh aspect is stored on a database.

Suitably, the localisation of said fusion protein in the method of the fifth to eleventh aspects of the invention is measured by its luminescence, fluorescence or radioactive properties.

Suitably, the agent induces activation of p38 Mitogen Activated Protein Kinase. Alternatively, the agent inhibits activation of p38 Mitogen Activated Protein Kinase.

Suitably, the agent is a chemical or physical entity. Preferably, the agent is a chemical which is a drug candidate. Preferably, the drug candidate is a pro- or anti-inflammatory compound.

### BRIEF DESCRIPTION OF THE DRAWINGS

FIG. 1A shows the DNA sequence of mitogen-activated protein kinase 14 (MAPK14).

FIG. 1B shows the protein sequence of mitogen-activated protein kinase 14 (MAPK14).

FIG. 2A illustrates a vector map of pCORON1002-EGFP-N1.

FIG. 2B illustrates a vector map of pCORON1002-EGFP-C1.

FIG. 3A shows a vector map for the adenoviral vector pDC515-UBC-Emerald-N1.

FIG. 3B shows a vector map for the adenoviral vector pDC515-UBC-Emerald-C1.

FIG. 4 is the protein sequence of c-terminal EGFP-p38 alpha (SEQ ID NO:3).

FIG. 5 is the protein sequence of n-terminal p38 alpha-EGFP (SEQ ID NO:4).

FIG. 6 is the protein sequence of c-terminal Emerald-p38 alpha (SEQ ID NO:5).

FIG. 7 is the protein sequence of n-terminal p38 alpha-Emerald (SEQ ID NO:6).

FIG. 8 is the DNA sequence of c-terminal EGFP-p38 alpha (SEQ ID NO:7).

FIG. 9 is the DNA sequence of n-terminal p38 alpha-EGFP (SEQ ID NO:8).

FIG. 10 is the DNA sequence of c-terminal Emerald-p38 alpha (SEQ ID NO:9).

FIG. 11 is the DNA sequence of n-terminal p38 alpha-Emerald (SEQ ID NO: 10).

FIG. 12 is a schematic diagram of a EGFP-MAPK14 translocation to the nucleus following activation.

FIG. 13A is an IN Cell Analyzer 3000 image of live SW1353 cells exhibiting stable expression of EGFP-C1-MAPK14.

FIG. 13B is an IN Cell Analyzer 3000 image of live SW1353 cells exhibiting stable expression of EGFP-C1-MAPK14 and their response to 0.4 M sorbitol treatment for 15 minutes.

FIG. 13C is an IN Cell Analyzer 3000 image of live SW1353 cells exhibiting stable expression of EGFP-C1-MAPK14 and their response to 300 nM anisomycin treatment for 15 minutes.

FIG. 14 is a graph of nuclear to cytoplasmic relocation (N:C ratio) of the EGFP-C1-MAPK14 fusion protein in living SW1353 cells against time. The time-lapse analysis was performed based upon imaging data acquired by an IN Cell Analyzer 3000 instrument. The graph shows the response of live cells to 300 nM anisomycin over 35 minutes (square symbol and continuous line) compared to untreated control cells (triangle symbol, dotted line).

FIG. 15A shows IN Cell Analyzer 3000 images of live SW1353 cells exhibiting stable expression of EGFP-C1-MAPK14 in response to 12 pM IL-1β treatment for 0 minutes.

FIG. 15B shows IN Cell Analyzer 3000 images of live SW1353 cells exhibiting stable expression of EGFP-C1-MAPK14 in response to 12 pM IL-1β treatment for 20 minutes.

FIG. 16 is a graph of nuclear to cytoplasmic relocation (N:C ratio) of the EGFP-C1-MAPK14 fusion protein in living SW1353 cells against time. The time-lapse analysis was performed based upon imaging data acquired by an IN Cell Analzyer 3000 instrument. The graph shows the response of live cells to IL-1β (12 pM) over 90 minutes (square symbol, continuous line) compared to untreated control cells (circle symbol, dotted line). Hoechst 33342 was not included in this experiment and no baseline response was visible in control cell (compare with FIG. 14).

FIG. 17 is an activator dose response curve produced with SW1353 cells exhibiting stable expression of the EGFP-C1-MAPK14 fusion protein treated for 30 minutes with IL-1β prior to fixation; analysis was based upon the nuclear to cytoplasmic ratio of the EGFP signal. Stable cells we cultured to passage 8 and 15 and the graph shows the stability of the response (MOR and EC50) with extended culture. Images and analysis were carried out on an IN Cell Analyzer 1000 instrument.

FIG. 18 is a plot of N:C ratio versus cell number per well for SW1353 cells exhibiting stable expression of the EGFP-C1-MAPK14 fusion protein treated for 30 minutes with IL-1β (12 pM) prior to fixation. Analysis was based upon the nuclear to cytoplasmic ratio of the EGFP signal obtained with data produced in ‘Repeatability of cytokine response assay’ above (n=144). Deviation of best fit lines from horizontal was significant for treated and untreated population (F=19 and 70, respectively; P<0.0001 for both curves). The images were acquired and analysed on an IN Cell Analyzer 1000 instrument. Symbol x and dotted line represents control cells and the circle symbol and continuous line represents data for IL-1β treated cells.

FIG. 19 shows images from a fixed cell assay showing response of SW1353 cells exhibiting stable expression of EGFP-MAPK14 to 316 pM IL-1β or control medium treatment for 30 minutes. Three colour images (Hoechst not shown) were taken on the IN Cell Analyzer 1000 and show EGFP-MAPK14 response (upper panels), Alexa 647 labelled antibody staining directed at phospho-p38 (middle panels) and co-localisation of signals (bottom panels).

FIG. 20 is an activator dose response curve produced with SW1353 cells exhibiting stable expression of the EGFP-C1-MAPK14 fusion protein (Green) treated for 30 minutes with IL-1β prior to fixation. After fixation the cells were treated with anti-phospho(Thr180/Tyr182)-p38(MAPK14) antibody and visualised with an Alexa647 labelled secondary antibody (Red). Analysis was based upon the nuclear to cytoplasmic ratio for the EGFP-MAPK14 signal (right Y axis, open circles and dotted line). Analysis was based upon nuclear-cytoplasmic intensity of the Alexa647-anti-phospho-p38 signal. EC₅₀ values of 7.16 and 7.82 pM (n=8) were obtained with the EGFP-MAPK14 and immunofluorescence assays, respectively.

FIG. 21 is an inhibitor response produced with SW1353 cells exhibiting stable expression of the EGFP-MAPK14 fusion protein incubated for 30 min with SB203580 (10 μM; n=8) prior to activation for 30 min with anisomycin (100 nM) and fixation. Bars are based upon the nuclear to cytoplasmic ratio of the EGFP signal. Images and analysis carried out on IN Cell Analyzer 1000 system.

FIG. 22 is an inhibitor dose response curve produced with SW1353 cells exhibiting stable expression of the EGFP-MAPK14 fusion protein incubated for 30 min with proprietary inhibitor A (0.005-300 nM; n=8) prior to activation for 30 min with IL-1β (12 pM =EC80) and fixation. The curve is based upon the nuclear to cytoplasmic ratio of the EGFP signal.

### DETAILED DESCRIPTION OF THE INVENTION

### Generation of MAPK14-reporter Gene Vectors

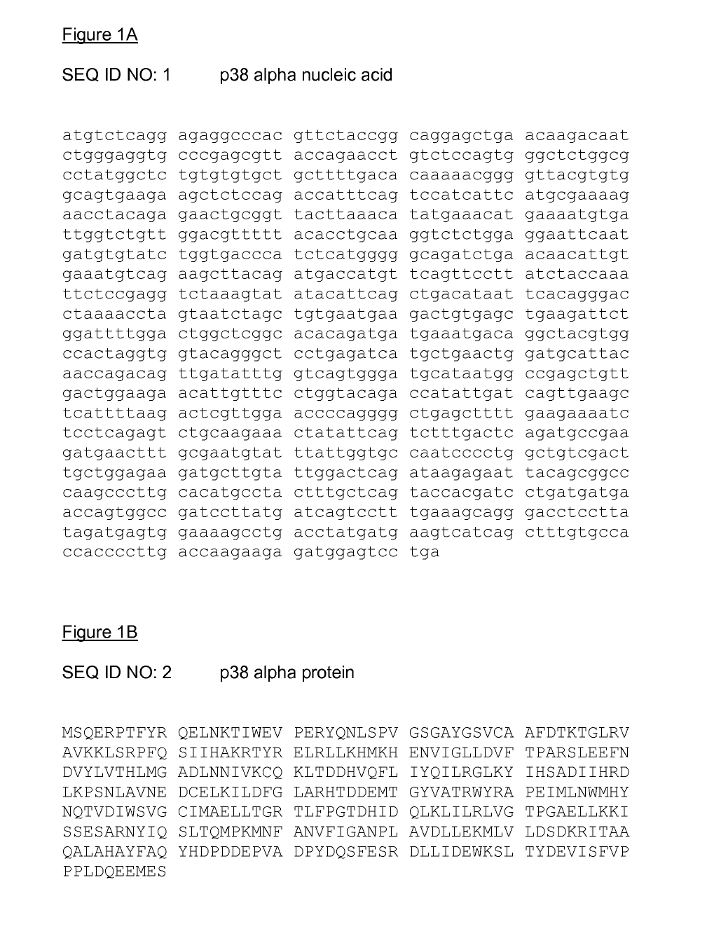

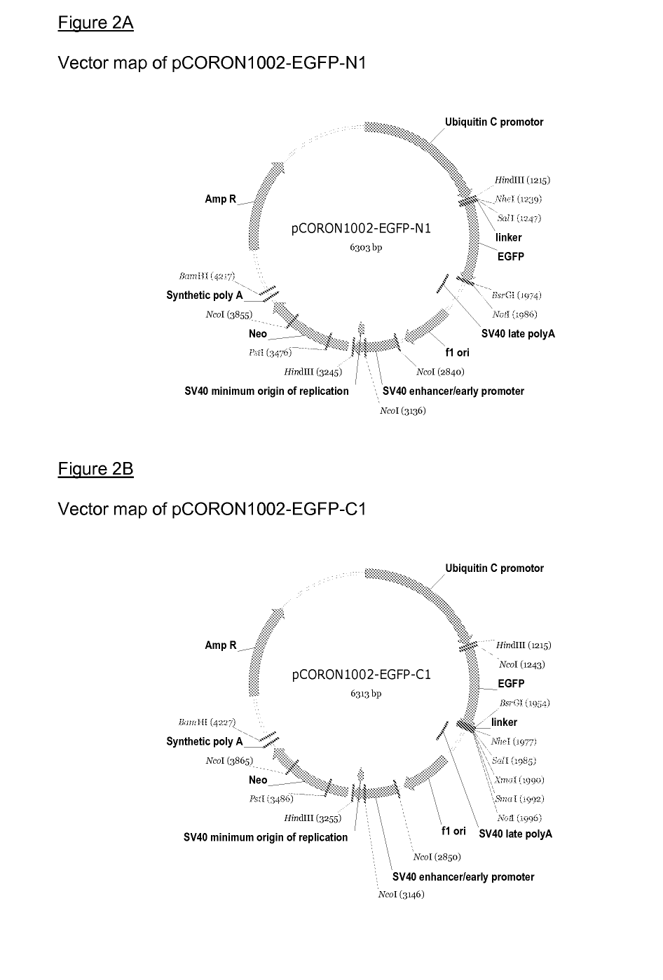

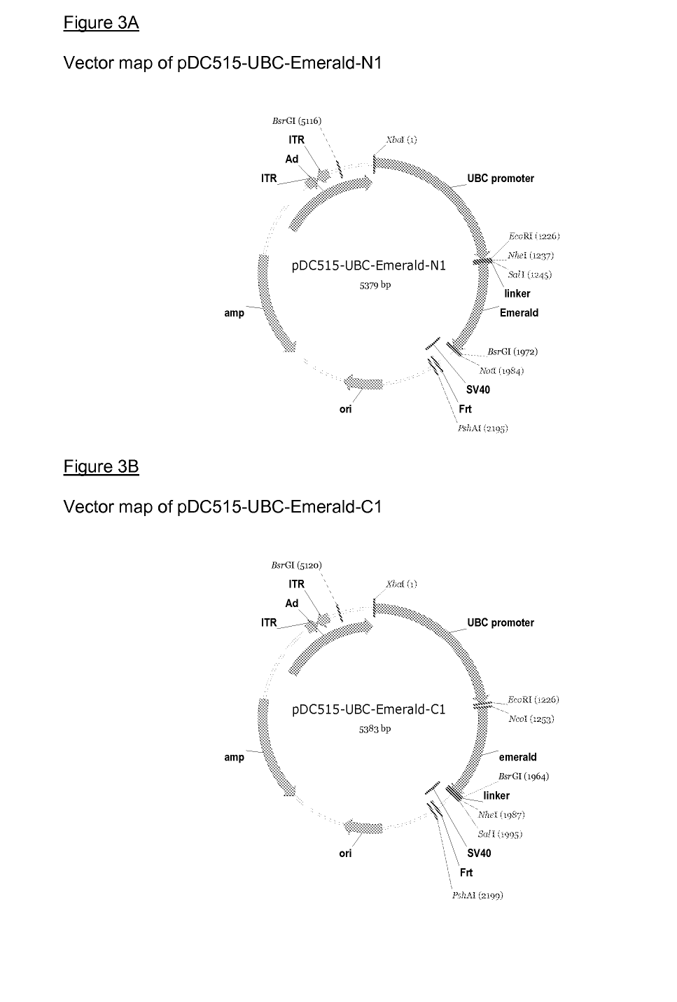

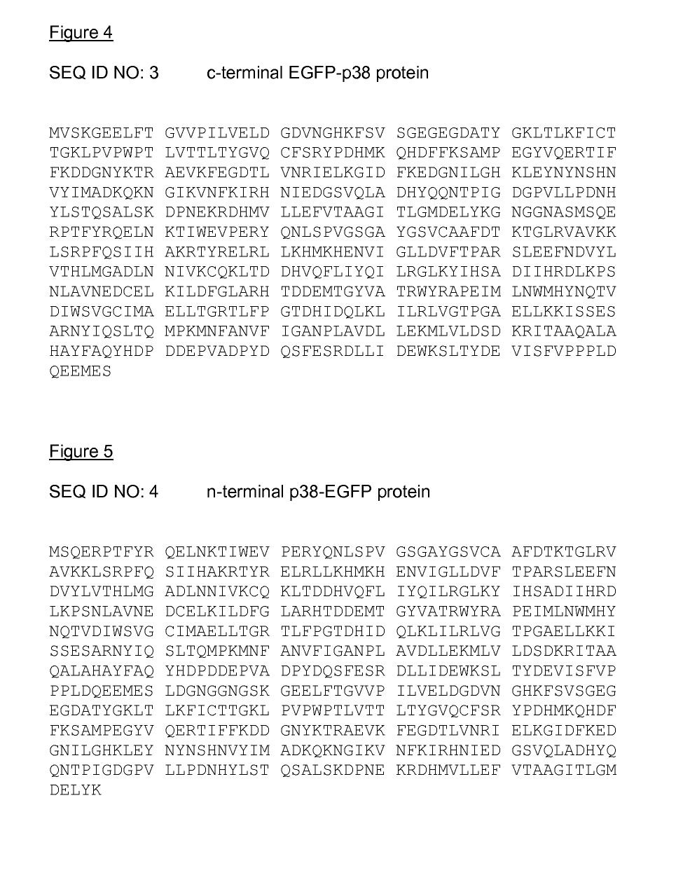

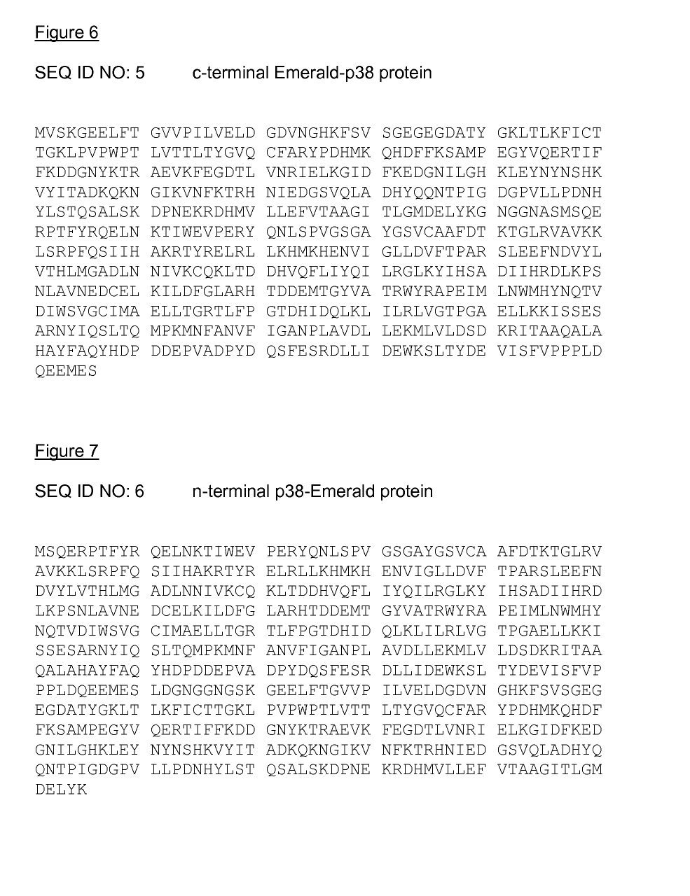

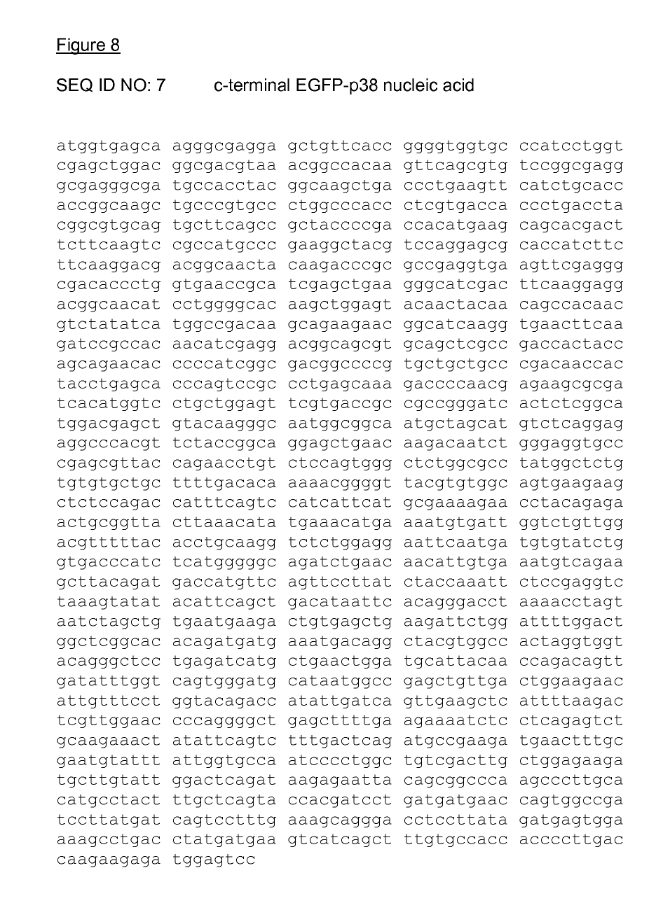

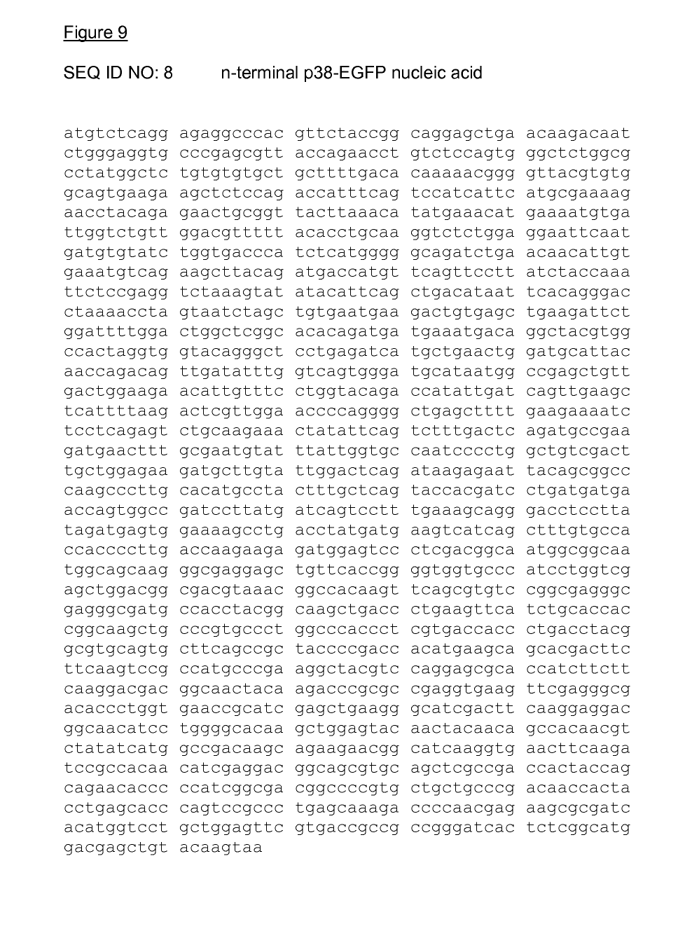

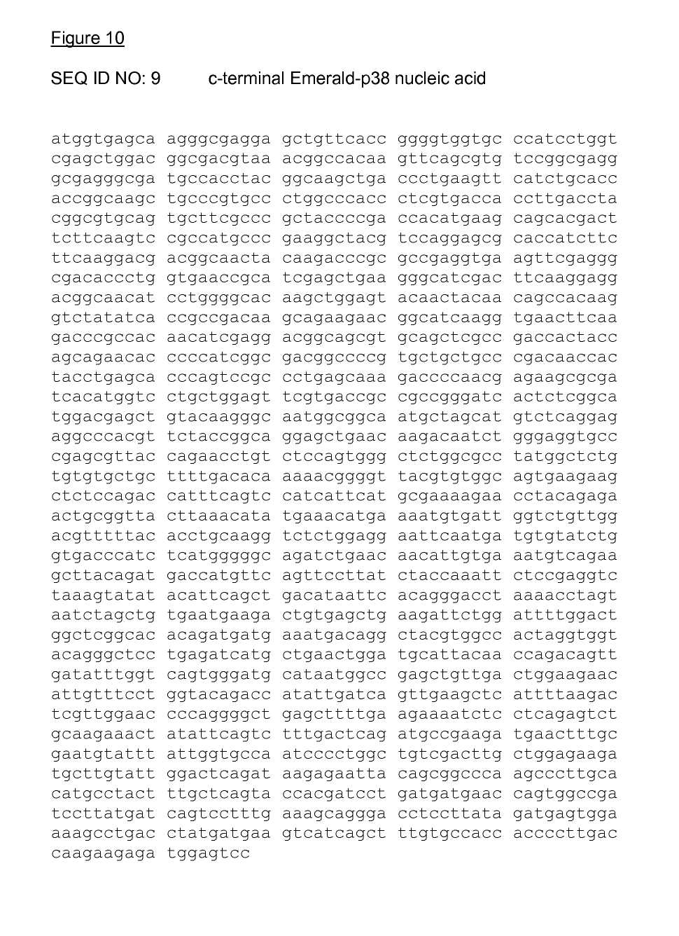

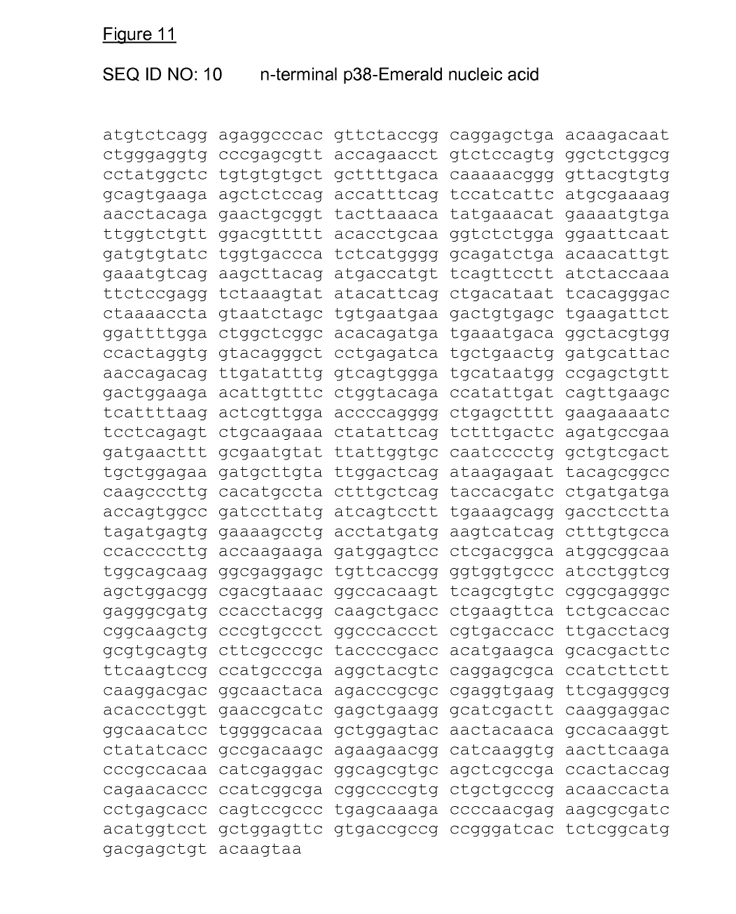

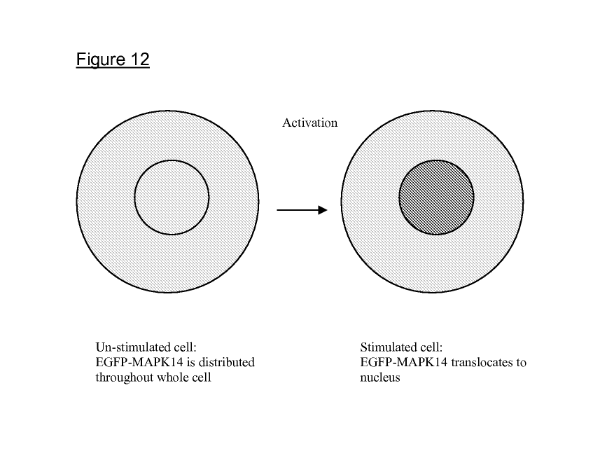

The gene corresponding to p38alpha MAPK14 variant 2 was obtained from the Mammalian Gene Collection (clone 5181064; GenBank BC031574; SEQ ID NO: 1, FIG. 1). SEQ ID NO: 2 shows the protein sequence encoded by SEQ ID NO: 1. PCR primers were designed to amplify the whole MAPK14 gene and permit the products to be subcloned as N-terminal or C-terminal fusions in plasmid vectors pCORON1002-EGFP-N1 and pCORON1002-EGFP-C1 (GE Healthcare, Amersham, UK; FIGS. 2a and 2b) or adenoviral vectors pDC515-UBC-Emerald-N1 and pDC515-UBC-Emerald-C1 (Microbix Biosystems Inc., Toronto, Calif.; FIGS. 3a and 3b). Introduction of a NheI restriction enzyme site at the 5′ end and XhoI restriction enzyme site at the 3′ end of the MAPK14 fragment allowed sub-cloning into NheI and SalI restriction sites in the vectors. The pCORON1002 vectors contain a bacterial ampicillin resistance gene and a mammalian neomycin resistance gene to facilitate stable cell line production; the fusion protein is expressed from the human ubiquitin C promoter. The human ubiquitin C promoter was chosen to produce homogeneous and consistent levels of fusion protein expression that are desirable in order to minimize perturbation of host cell systems and produce a cell line and assay that are stable and robust.

### Generation of Cell Line Exhibiting Stable Expression of the EGFP-MAPK14 Fusion Protein

The human chondrosarcoma cell line SW1353 (ATCC) was transfected with the plasmid vectors pCORON1002 EGFP-C1-MAPK14 and pCORON1002 EGFP-N1-MAPK14 using FuGENE 6 transfection reagent (Roche, UK). Cells were maintained in RPMI 1640 medium (Sigma-Aldrich, UK) supplemented with 10% (v/v) FBS, 1% (v/v) penicillin-streptomycin, 1% (v/v) glutamine. Stable clones expressing the recombinant fusion protein were selected over 4 weeks by selection in geneticin (500 μg/ml). Primary clone characterisation at early passage involved flow cytometry to determine homogeneity and level of expression (FACSCalibur; BD Biosciences), morphological relevance of cells compared to the parental cell line and physiological relevance of biological response. These characteristics were then analysed at later passage to assess temporal stability of the cell line. Secondary clones were isolated where appropriate and analysed in a similar manner. Cell lines that exhibited stable expression of the EGFP-MAPK14 fusion protein and met desired criteria were maintained using growth medium containing 200 μg/ml geneticin. Stable expression of the fusion protein has been demonstrated to passage 30 for selected clones.

### Assays for Activation of MAPK14 by Measurement of Fusion Protein Translocation Using a Cell Line Exhibiting Stable Expression of EGFP-MAPK14

The EGFP-MAPK14 fusion protein population resides predominantly in the cytoplasm of, or is distributed evenly throughout, resting cells. However, when cells are stimulated through external stress such as osmotic shock or protein translation inhibition, or through treatment with cytokines, a proportion of the MAPK14 labelled population relocalises to the nucleus (FIG. 12).

During assay preparation and throughout the assay, background stress response was kept to a minimum; microplates, cells and solutions were maintained at 37° C., 5% CO₂, and 95% relative humidity. All inhibitors and activators were added to cells in complete medium.

Cells exhibiting stable expression of the EGFP-MAPK14 fusion were seeded at 8×103 cells per well in 100 μl maintenance medium in Packard Black 96 Well ViewPlates. 50 μl of the prepared test activator in complete medium and control compounds were added and incubated at 37° C., 5% CO2, and 95% relative humidity. For fixed cell assays, 150 μl 10% (v/v) formalin (4% formaldehyde) was added at room temperature for 20 min. Cells were washed twice with PBS and nuclei were stained with Hoechst (10 μM) in PBS prior to imaging. Plates were viewed on IN Cell Analyzer 1000 (GE Healthcare, Amersham, UK) or IN Cell Analyzer 3000 (GE Healthcare, Amersham, UK) and images were analysed with the IN Cell Analyzer 1000 Nuclear Translocation Analysis Module (GE Healthcare, Amersham, UK) or IN Cell Analyzer 1000 Morphology Analysis Module (GE Healthcare, Amersham, UK) according to the manufacturers instructions.

### Response to Osmotic Shock

Living SW1353 cells which exhibited stable expression of EGFP-C1-MAPK14 were treated with 0.4M sorbitol or 300 nM anisomycin for 15 minutes and their response compared to untreated, control cells expressing the same construct.

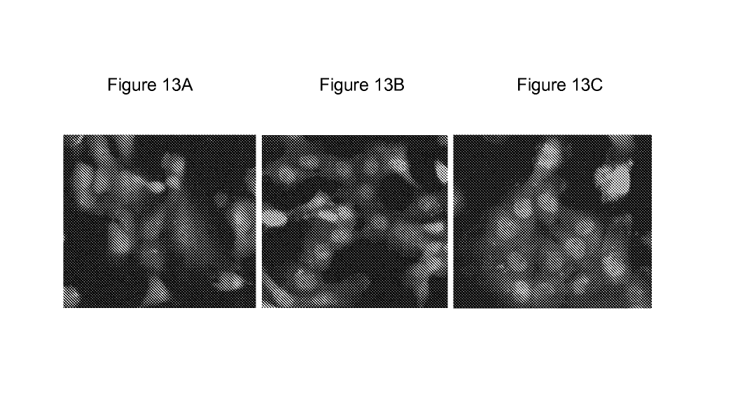

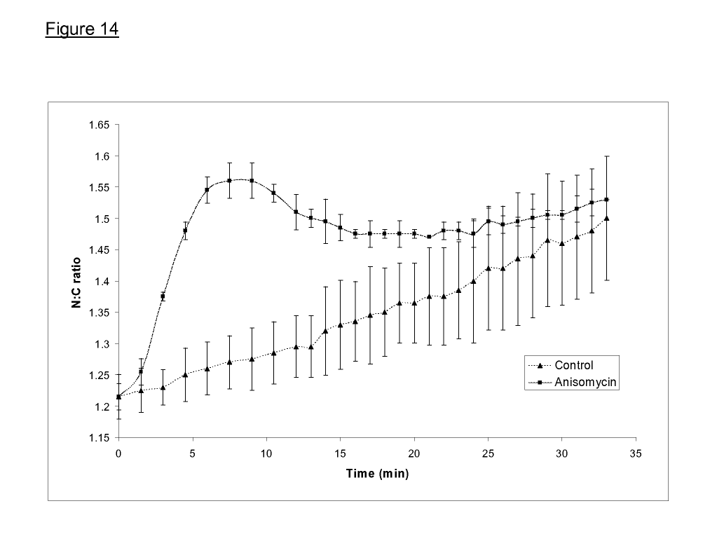

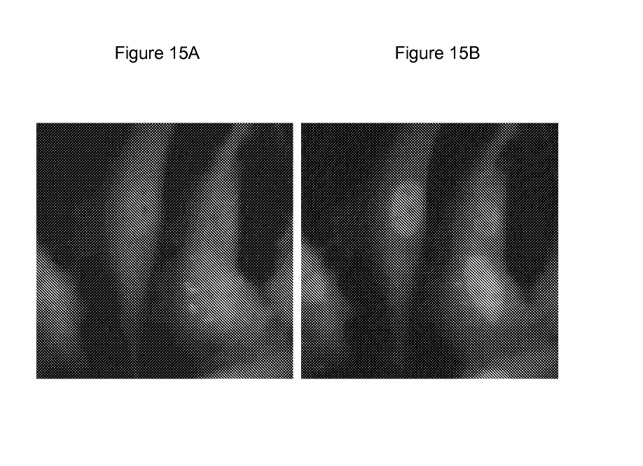

Images were acquired of live SW1353 cells stably expressing EGFP-C1-MAPK14 using an IN Cell Analyzer 3000 instrument and are shown in FIG. 13. The left panel (FIG. 13A) shows images of cells after 15 minutes in the medium containing no activators or inhibitors (i.e. ‘control cells’), where the green fluorescence of the reporter protein is seen throughout each cell. The translocation of EGFP-MAPK14 to the nucleus in response to treatment with 0.4 M sorbitol (middle panel, FIG. 13B) or 300 nM anisomycin (right panel, FIG. 13C) treatment for 15 minutes can be seen in FIGS. 13B and 13C.

Cells treated with 0.4M sorbitol for 15 mins clearly demonstrate nuclear accumulation of the EGFP-MAPK14 fusion protein and this stimulation is evident until at least 90 minutes after initial exposure.

### Response to Protein Translation Inhibition

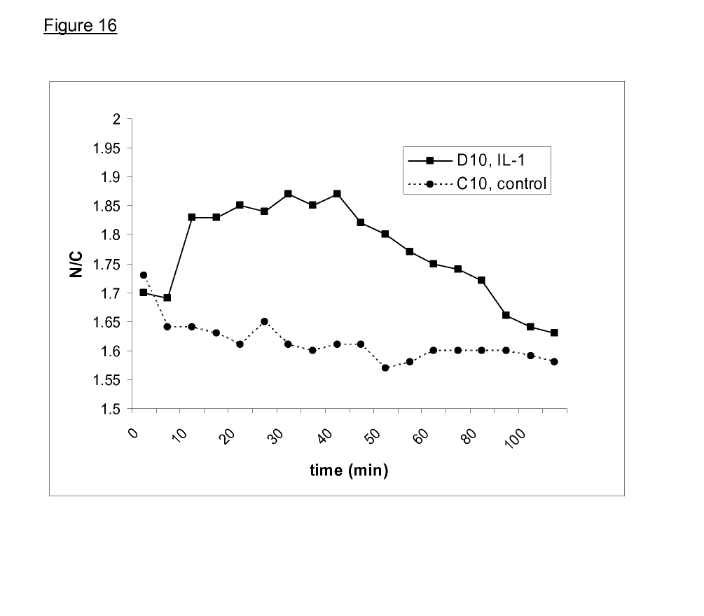

An analysis of live-cell time lapse images from IN Cell Analyzer 3000 data (FIG. 14) shows the significant nuclear to cytoplasmic relocation (N:C ratio) of the EGFP-C1-MAPK14 fusion protein in SW1353 cells in response to anisomycin (300 nM) over 35 minutes. Hoechst 33342 was included in this experiment and has caused a baseline stress response in control cells.

Cells treated with 300 nM anisomycin for 15 mins clearly demonstrate significant nuclear accumulation of the EGFP-MAPK14 fusion protein (FIGS. 13C and 14). However, time lapse analysis indicates that, as would be expected with a stress response of this type, there is a gradual equilibration of the signal between 20 and 35 minutes (FIG. 14).

### Response of SW1353 Cells to Cytokines

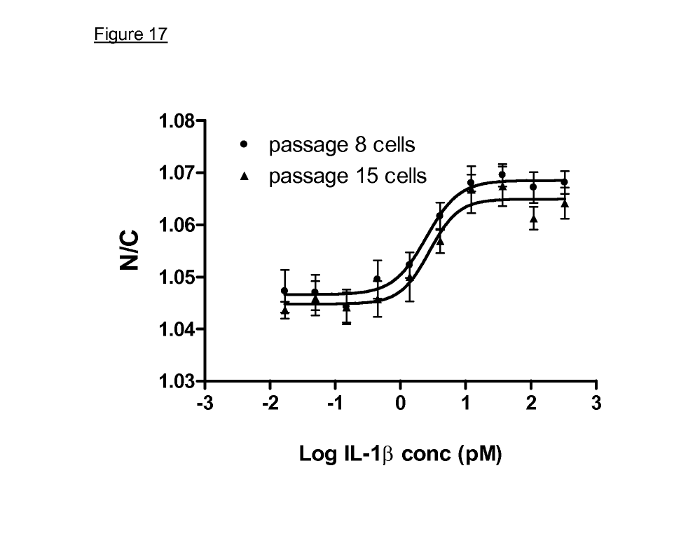

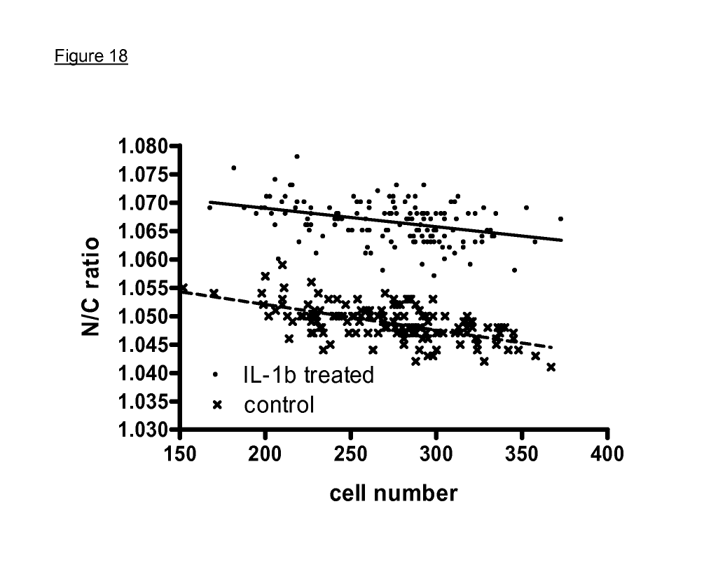

Living cells treated with 12 pM IL-1β show a clear translocation of EGFP-MAPK14 signal from cytoplasm to nucleus (FIGS. 15A and 15B) with maximum accumulation of signal in the nucleus between 20 and 40 min (FIG. 16). As would be expected for an inflammatory response there is a gradual equilibration of the signal between 40 and 90 min (FIG. 16).

### Cytokine Dose Response

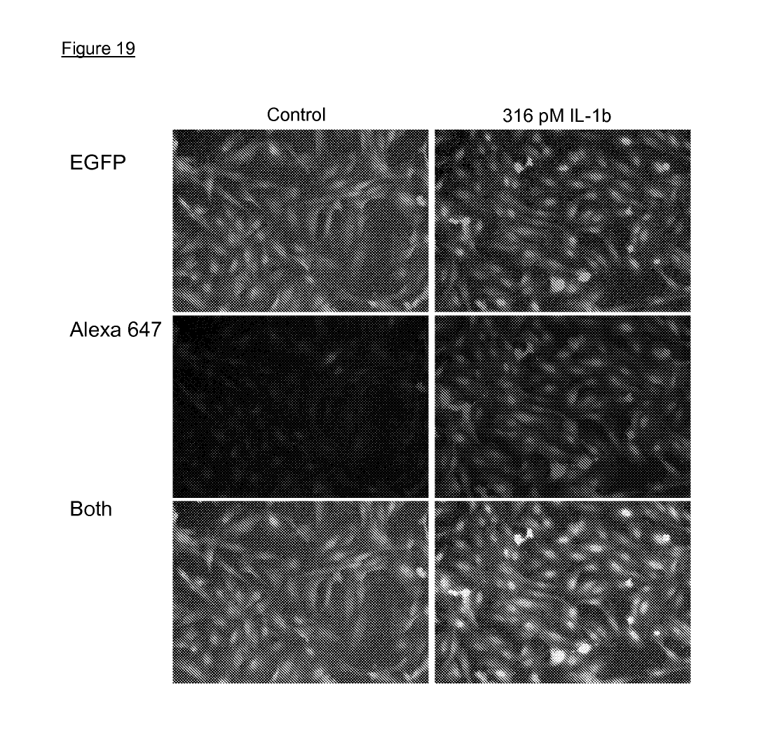

IL-1β was added to SW1353 cells exhibiting stable expression of EGFP-C1-MAPK14 to a final concentration between 0.017-333 pM. Dose response curves for cells at passage 8 and 15 of one clonal stable cell population show the temporally robust nature of the response of the particular cell line (FIG. 17). EC50 values of 2.5 and 2.8 pM, respectively were obtained (N=8, S:N=6.5 and 4, respectively).

### Repeatability of Cytokine Response Assay

A study of the repeatability of the assay with respect to translocation of the EGFP-MAPK14 fusion protein within SW1353 cells exhibiting stable expression was carried out. Assays were run on 3 separate 96-well plates. Alternate wells of each plate contained cells treated with control medium (n=48) or IL-1β (12 pM). The overall signal to noise value was 3.61:1 (n=144).

### Variation of Cytokine Response Assay With Cell Seeding Density and Growth Rate

Isolation of the variation that contributes to the standard deviation in the mean of nuclear to cytoplasmic ratio for treated and untreated cells would facilitate the production of an improved assay for screening compounds that activate or inhibit p38 MAPK signalling.

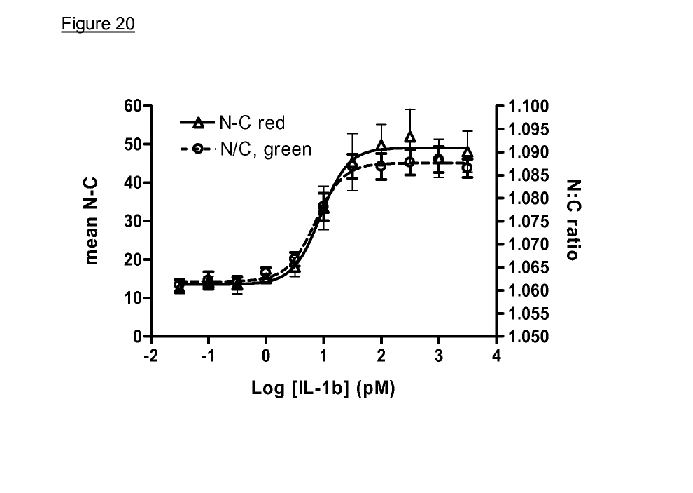

When the N:C ratio of cells treated with IL-1β or control medium (data from ‘Repeatability of cytokine response assay’ above) was analysed with respect to cell number it is clear that cell seeding density and cell number contribute significantly toward assay variation, since the ‘best fit’ lines are not horizontal (FIG. 18). It is extremely difficult to deposit exactly the same number of cells into every well during a screen or control growth rate across a plate due to edge effects etc. Therefore, in a drug screening assay the use of an N:C ratio versus cell number plot facilitates the differentiation of true ‘hits’, since effects due to variation in cell number, toxicity and cell cycle arrest caused by compounds will be evident.

### Correlation Between Biological Activation of EGFP-MAPK14 Fusion Protein and Anti-phospho-p38 Immunofluorescence Assay

The temporal and biological response of the EGFP-MAPK14 assay was validated through co-analysis with a recognised assay for p38 (MAPK14) activation—an immunofluorescence assay targeting phospho-(Thr180/Tyr182)-p38(MAPK14). SW1353 cells exhibiting stable expression of the EGFP-MAPK14 fusion protein were treated with IL-1β and fixed and stained with Hoechst as described above. Cells were then washed (1% goat serum, 0.1% Tween in PBS), permeabilized (0.5% Triton X in wash buffer) for 15 minutes at room temperature and washed again. 50 μl of rabbit anti-phospho-p38 antibody at 1:200 (Zymed Laboratories, San Francisco, USA) was added and incubated for 1 hr at room temperature prior to two washes and the addition of 50 μl of goat-anti rabbit Alexa647 antibody (Molecular Probes) at 1:200 and incubation for 1 hr at room temperature. After 2 washes, cells were imaged in PBS to detect Hoechst, EGFP-MAPK14 and Alexa647 on an IN Cell Analyzer 1000 instrument.

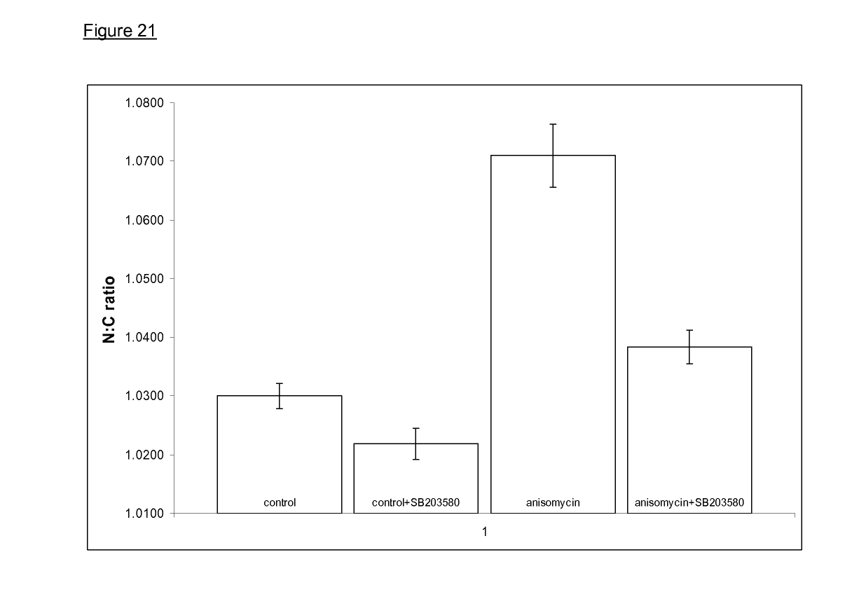

Cells treated with IL-1β showed a clear translocation of the green EGFP-MAPK14 signal from cytoplasm to nucleus (FIG. 19). In cells treated with IL-1β the phospho-p38 signal detected with the immunofluorescence assay (Red signal) and the EGFP-MAPK14 signal co-localised demonstrating that these methods correlate and either or both methods can be used to detect activation of MAPK14 (FIG. 19). In addition, employing both methods may permit differentiation of compounds that activate/inhibit phosphorylation but not translocation. A dose response curve produced using both methods showed a good correlation between the methods (FIG. 20). EC50 values of 7.16 and 7.82 pM were obtained with the EGFP-MAPK14 and immunofluorescence assays, respectively

### Assays for Inhibitors of Activation of MAPK14 by Measurement of Fusion Protein Translocation Using a Stable Cell Line Exhibiting Expression of EGFP-MAPK14

Cells exhibiting stable expression of the EGFP-MAPK14 fusion protein were seeded at 0.8×10⁴ cells per well in 100 μl maintenance medium in Packard Black 96 Well ViewPlates. Cells were then pre-incubated by addition 25 μl of inhibitor in medium for 30 min prior to addition of 25 μl of prepared test activator or control in medium and incubated for a further 30 mins. Cells were fixed, imaged and analysed as described above.

The activation of MAPK14 and translocation of the fusion protein in SW1353 cells by anisomycin (100 nM) was significantly inhibited by pre-incubation with the p38α (MAPK14) inhibitor SB203580 (10 μM; FIG. 21).

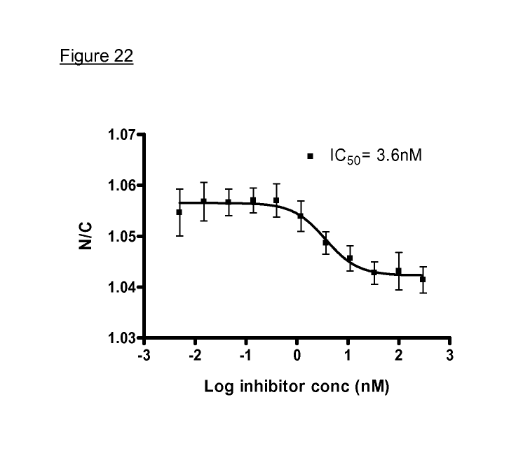

Cells that were pre-incubated with a proprietary inhibitor of IL-1β induced activation of MAPK14 clearly did not respond to activation and an EC₅₀ of 3.6 nM was produced (FIG. 22).

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A fusion protein comprising a reporter gene product and an isoform of p38 Mitogen Activated Protein Kinase (MAPK)

2. The fusion protein of claim 1, additionally comprising a linker group linking said reporter gene product to said p38 MAPK.

3. The fusion protein of claim 2, wherein said linker group consists of a peptide comprising less than ten peptides.

4. The fusion protein of claim 3, wherein said linker group consists of the amino acids GNGGNAS.

5. The fusion protein of claim 1, wherein the isoform of p38 MAPK is selected from the group consisting of p38-alpha (p38α, MAPK14), p38-beta (p38β), p38δ (SAPK4) and p38 gamma (p38γ or ERK6, SAPK3).

6. The fusion protein of claim 5, wherein the isoform of p38 MAPK is p38-alpha (MAPK14).

7. The fusion protein of claim 1, wherein the reporter gene product is localisable by a detectable luminescent, fluorescent or radio-active moiety.

8. The fusion protein of claim 1, wherein the reporter gene product is a fluorescent protein.

9. The fusion protein of claim 8, wherein said fluorescent protein is selected from the group consisting of Green Fluorescent Protein (GFP), Yellow Fluorescent Protein (YFP), Blue Fluorescent Protein (BFP), Cyan Fluorescent Protein (CFP), Red Fluorescent Protein (RFP), Enhanced Green Fluorescent Protein (EGFP) and Emerald.

10. The fusion protein of claim 1, comprising Enhanced Green Fluorescent Protein and p38-alpha (MAPK14).

11. The fusion protein of claim 1, comprising Emerald and p38-alpha (MAPK14).

12. The fusion protein of claim 1, selected from the group consisting of SEQ ID NO: 3, SEQ ID NO: 4, SEQ ID NO: 5 and SEQ ID NO: 6.

13. A nucleotide sequence encoding a fusion protein of claim 1.

14. The nucleotide sequence of claim 13, selected from the group consisting of SEQ ID NO: 7, SEQ ID NO: 8, SEQ ID NO: 9 and SEQ ID NO: 10.

15. A nucleotide sequence of claim 13, wherein said sequence is operably linked to a promoter and is under the control of said promoter.

16. The nucleotide sequence of claim 15, wherein said promoter is selected from the group consisting of mammalian constitutive promoter, mammalian regulatory promoter, human ubiquitin C promoter, viral promoter, SV40 promoter, CMV promoter, yeast promoter, filamentous fungal promoter and bacterial promoter.

17. The nucleotide sequence of claim 16, wherein said viral promoter is the CMV or the SV40 promoter.

18. The nucleotide sequence of claim 16, wherein the promoter is the human ubiquitin C promoter.

19. A replicable vector comprising a nucleotide sequence of claim 13.

20. The replicable vector of claim 19, wherein said vector is a plasmid vector.

21. The replicable vector of claim 19, wherein the vector is a viral vector.

22. The replicable vector of claim 22, wherein said viral vector is selected from the group consisting of cytomegalovirus, Herpes simplex virus, Epstein-Barr virus, Simian virus 40, Bovine papillomavirus, Adeno-associated virus, Adenovirus, Vaccina virus and Baculovirus vector.

23. A host cell transformed with a nucleotide sequence of claim 13.

24. A host cell according to claim 23, wherein said nucleotide sequence is stably transformed in said host cell.

25. The host cell of claim 23, selected from the group consisting of plant, insect, nematode, bird, fish and mammalian cell.

26. The host cell of claim 25, wherein said mammalian cell is a human cell.

27. The host cell of claim 26, wherein said human cell is the human chondrosarcoma cell line SW1353.

28. The host cell of claim 13 capable of expressing said fusion protein.

29. A method for detecting activation of p38 Mitogen Activated Protein Kinase (MAPK) in a living cell comprising the steps of:
i) culturing a cell transformed to over-express a fusion protein comprising a reporter gene product and an isoform of p38 Mitogen Activated Protein Kinase;
ii) determining the localisation of the fusion protein within the cell with time;
wherein a change in localisation of the fusion protein within the cell is indicative of activation.

30. A method for measuring the effect that an agent has upon activating p38 Mitogen Activated Protein Kinase (MAPK) in a living cell comprising the steps of:
i) culturing a cell transformed to over-express a fusion peptide comprising a reporter gene product and an isoform of p38 Mitogen Activated Protein Kinase;
ii) determining the localisation of said peptide within the cell;
ii) treating the cell with said agent and determining the localisation of the peptide within the cell;
wherein any difference in the localisation of the peptide within the cell relative to control cells untreated with the agent is indicative of the effect that the agent has upon activating p38 Mitogen Activated Protein Kinase (MAPK).

31. A method for measuring the effect an agent has upon activating p38 Mitogen Activated Protein Kinase (MAPK) in a living cell comprising the steps of:
i) culturing a first cell and a second cell which both over-express a fusion protein comprising a reporter gene product and an isoform of p38 Mitogen Activated Protein Kinase;
ii) treating said first cell with said agent and determining the localisation of said protein within the first cell;
iii) determining the localisation of the protein within said second cell which has not been treated with the agent;
wherein any difference in the localisation of the protein within the first cell and second cell is indicative of the effect that the agent has upon activating p38 Mitogen Activated Protein Kinase (MAPK).

32. A method for measuring the effect an agent has upon activating p38 Mitogen Activated Protein Kinase (MAPK) in a living cell comprising the steps of:
i) culturing a cell transformed to over-express a fusion protein comprising a reporter gene product and an isoform of p38 Mitogen Activated Protein Kinase;
ii) treating said cell with said agent and determining the localisation of the protein within the cell;
iii) comparing the localisation of the protein in the presence of the agent with a known value for the localisation of the protein in the absence of the agent;
wherein any difference in the localisation of the protein within the cell in the presence of the agent and said known value in the absence of the agent is indicative of the effect that the agent has upon activating p38 Mitogen Activated Protein Kinase (MAPK).

33. A method for measuring the effect that an agent has upon modulating the activation of p38 Mitogen Activated Protein Kinase (MAPK) in a living cell comprising the steps of:
i) culturing a cell transformed to over-express a fusion peptide comprising a reporter gene product and an isoform of p38 Mitogen Activated Protein Kinase;
ii) treating the cell with said agent;
iii) treating the cell with a known activator of p38 Mitogen Activated Protein Kinase (MAPK) and determining the localisation of the peptide within the cell;
wherein any difference in the localisation of the peptide within the cell relative to control cells untreated with the agent but treated with said known activator is indicative of the effect that the agent has upon modulating activation of p38 Mitogen Activated Protein Kinase (MAPK).

34. A method for measuring the effect an agent has upon modulating the activation of p38 Mitogen Activated Protein Kinase (MAPK) in a living cell comprising the steps of:
i) culturing a first cell and a second cell which both over-express a fusion protein comprising a reporter gene product and an isoform of p38 Mitogen Activated Protein Kinase;
ii) treating said first cell with said agent;
iii) treating the first cell and the second cell with a known activator of p38 Mitogen Activated Protein Kinase (MAPK) and determining localisation of the protein within the first cell and the second cell;
wherein any difference in the localisation of the protein within the first cell and second cell is indicative of the effect that the agent has upon modulating activation of p38 Mitogen Activated Protein Kinase (MAPK).

35. A method for measuring the effect an agent has upon modulating the activation of p38 Mitogen Activated Protein Kinase (MAPK) in a living cell comprising the steps of:
i) culturing a cell transformed to over-express a fusion protein comprising a reporter gene product and an isoform of p38 Mitogen Activated Protein Kinase;
ii) treating said cell with said agent;
iii) treating the cell with a known activator of p38 Mitogen Activated Protein Kinase (MAPK) and determining the localisation of the peptide within the cell;
iv) comparing the localisation of the protein in the presence of the agent and said activator with a known value for the localisation of the protein in the presence of the activator but in the absence of the agent;
wherein any difference in the localisation of the protein within the cell in the presence of the agent and said known value in the absence of the agent is indicative of the effect that the agent has upon modulating the activation of p38 Mitogen Activated Protein Kinase (MAPK).

36-39. (canceled)

40. The method of claim 30, where the agent is a chemical or physical entity.

41. The method according to claim 40, wherein said chemical is a drug candidate.

42. The method according to claim 41, wherein said drug candidate is a pro- or anti-inflammatory compound.
